(19) United States
(12) Patent Application Publication
(10) Pub. No.: US 2026/0345473 A1
(43) Pub. Date: Nov. 5, 2026

(54) Fermented Arnica Oil in Personal Care Applications

(71) Applicant: HLB90067, Inc., El Segundo, CA (US)

(72) Inventors: Stefani Germanotta, Malibu, CA (US); Gloria Ryu, Irvine, CA (US); Susan Goldsberry, Newport Coast, CA (US)

(73) Assignee: HLB90067, Inc., El Segundo, CA (US)

(21) Appl. No.: 19/110,138

(22) PCT Filed: Sep. 8, 2023
(86) PCT No.: PCT/US2023/032262

§ 371 Date: Mar. 10, 2025

Related U.S. Application Data
(60) Provisional application No. 63/404,952, filed on Sep. 8, 2022.

(51) Int. Cl. A61K 8/9789; A61K 31/728; A61K 36/232; A61K 36/28; A61K 36/30; A61K 36/484; A61P 17/00; A61P 29/00; A61Q 17/00; A61Q 19/02; A61Q 19/08

(52) U.S. Cl. CPC A61K 8/9789; A61K 31/728; A61K 36/232; A61K 36/28; A61K 36/30; A61K 36/484; A61P 17/00; A61P 29/00; A61Q 17/00; A61Q 19/02; A61Q 19/08; A61K 2236/19; A61K 2800/85

(57) ABSTRACT

An anti-inflammatory fermented arnica oil produced by fermenting oil extracted from the flowers of A. montana with a strain of Pseudozyma spp., preferably Pseudozyma epicula; compositions containing said fermented arnica oil or a complex containing said fermented arnica in combination with one or more secondary fermented oils that provides skin benefits including reduction of under-eye bag area, reduction of dark circles and/or puffiness in the under-eye area, increased skin brightness, increased skin hydration, reduced transepidermal water loss, improved skin barrier integrity.

### BACKGROUND OF THE INVENTION

Inflammation is a multi-step process that occurs after disruption of tissue homeostasis by cell stress, injury or infection, and involves recruitment and retention of cells to affected sites to resolve damage and initiate repair. Nuclear factor kappa-light-chain-enhancer of activated B cells (NFκ-B) acts as a master regulator, mediating an array of genes involved in different stages of inflammation-including interleukins and tumor necrosis factor alpha (TNF-α), cytokines that activate the inflammatory process.

Among the most common stressors that elicit inflammatory responses in skin cells is solar radiation. UVB irradiation induces expression of tumor necrosis factor alpha (TNF-α) in both keratinocytes and dermal fibroblasts. Bashir et al. “TNF-alpha production in the skin” Arch Dermatol Res. 2009 January; 301(1):87-91. UVA radiation deeply penetrates the skin, resulting in a “cascade impairment of cell functions and tissue degradation.” Battie, C. et al., Exp Dermatol. “New insights in photoaging, UVA induced damage and skin types.” 2014 October; 23 Suppl 1:7-12. These negative sequelae are evidenced initially at the cellular level—in modulation of gene expression—and later as visible changes in the appearance of the skin. UVA-induced photoaging is seen clinically not only in terms of increased number and depth of facial fine lines and/or wrinkles and/or uneven pigmentation, but also reduced barrier function which can be measured in terms of loss of elasticity and/or decreased hydration.

Reactive oxygen containing species (ROS)-peroxyl radicals, superoxide radicals and hydroxyl radicals—are generated during a variety of stresses on cells (e.g., exposure to ultraviolet radiation) and produce inflammation and tissue damage, including oxidation of polyunsaturated lipids in cell membranes. Attack by free radicals on carbon-to-carbon double bonds lead to the formation of ROS and the initiation of a chain reaction in which the end products, lipid peroxides, compromise cell membrane integrity. Ames B N et al. “Oxidants, antioxidants, and the degenerative diseases of aging.” Proc Natl. Acad. Sci. U.S.A. 1993, 90:7915-22.

The antioxidant activity of a material (e.g., an individual ingredient, a complex of ingredients) on generation of ROS can be assessed using fluorescence or luminescence. One commonly used metric is Oxygen Radical Absorbance Capacity (ORAC), which quantifies the capacity of oxygen radicals to damage skin cells. More particularly, ORAC measures the effect of a test material on oxidative degradation of fluorescein after being mixed with a peroxyl radical. Antioxidant materials reduce the amount of fluorescence. ORAC values are expressed relative to a standard antioxidant (Trolox, a vitamin E analogue).

A variety of methods are known in the art to quantify total ORAC, as well as ORAC for lipophilic and hydrophilic components of a test material. One lipo-ORAC assay uses methylated beta-cyclodextrin as a solubility enhancer and measures the ability of lipophilic antioxidant compounds to inhibit the decline in disodium fluorescein fluorescence induced by a peroxyl radical generator, 2′,2′-Azobis (2-amidinopropane) dihydrochloride (AAPH). Huang, D. et al “Development and Validation of Oxygen Radical Absorbance Capacity Assay for Lipophilic Antioxidants using Randomly Methylated-Cyclodextrin as the Solubility Enhancer” J. Agric. Food Chem. 2002, 50(7): 1815-21.

Other antioxidant test methods known to persons having ordinary skill in the art include: Trolox equivalent antioxidant capacity assay (TEAC I-III assay), Total radical-trapping antioxidant parameter assay (TRAP assay), 2,2-diphenyl-l-picrylhydrazyl assay (DPPH assay), N,N-dimethyl-p-phenylendiamine assay (DMPD assay), Photochemiluminescence assay (PCL assay) and Ferric reducing ability of plasma assay (FRAP assay). K. Schleiser et al, “Assessment of antioxidant activity by using different in vitro methods” Free. Radic. Res. 2002 February; 36(2):177-87; Cao et al. “Oxygen-radical absorbance capacity assay for antioxidants” Free Radic. Biol. Med. 1993 March; 14(3):303-11; Analysis By Emitted Light (ABEL) assay kit (Knight Scientific Limited Plymouth, United Kingdom). See also U.S. Patent Application Pre-Grant Publication Nos. 2004/0109905 and 2005/0163880.

In inflammatory skin conditions, including atopic dermatitis, cytokines are upregulated, and skin barrier function is disrupted. Danso et al. “TNF-α and Th2 Cytokines Induce Atopic Dermatitis-Like Features on Epidermal Differentiation Proteins and Stratum Corneum Lipids in Human Skin Equivalents.” J. Invest. Dermatol. 2014; 134:1941-1950.

Loricrin (LOR) is an important protein for skin barrier formation and integrity. In studies of atopic dermatitis and other inflammatory skin conditions, the scientific literature reports a significant decrease in the expression of the loricrin gene (and consequently levels of loricrin protein in upper layers of the epidermis responsible for barrier protection). Furue, M. “Regulation of Filaggrin, Loricrin, and Involucrin by IL-4, IL-13, IL-17A, IL-22, AHR, and NRF2: Pathogenic Implications in Atopic Dermatitis.” Int. J. Mol. Sci. 2020 Jul. 29; 21(15):5382.

Fermented oils are marketed as superior to conventional oils in several respects—improved aesthetics (less greasy feel), better skin absorption, spreadability, and increased shelf life; and are described in the following patent publications:

International Application PCT/KR2022/007832 published as WO/2023/277367 teaches a cosmetic composition comprising, as active ingredients, a Pseudozyma antarctica strain and a Pseudoalteromonas neustonica strain (or a fermentation solution, lysate, extract or culture medium of either or both of the two strains).

Korean Patent No. 10-1974103 teaches a cosmetic composition comprising horse fat fermented by Pseudozyma sp. SY16 (KCTC 8950P; Korean Collection for Type Cultures (KCTC), Biological Resource Center of the Korea Research Institute of Bioscience and Biotechnology).

Korean Patent No. 10-1715652 teaches a process for making fermented oils containing nitro fatty acid involving fermentation of vegetables oils (olive oil, palm oil, argan oil, green tea seed oil, grape seed oil, meadowfoam seed oil, soybean oil, sunflower seed oil, grape seed oil, sesame oil, rapeseed oil, canola oil, shoulder oil, flax oil, safflower seed oil, corn oil, rapeseed oil, canola oil) with Pseudozyma sp. SY16 and nitric acid bacterium.

Korean Patent No. 10-1716680 teaches fermenting ingredients in Shiunko, Lithospermum erythrorhizon and Korean angelica (Angelica gigas) root extract, in a vegetable oil (olive oil, palm oil, argan oil, green tea seed oil, grape seed oil, meadowfoam seed oil, soybean oil, sunflower seed oil, grape seed oil, sesame oil, shoulder oil, flax oil, safflower seed oil, corn oil, rapeseed oil, and canola oil) with a glycolipid-biosurfactant-producing microorganism—a Pseudozyma microorganism, specifically Pseudozyma sp. SY16.

Korean Patent No. 10-2490747 teaches fermentation of a vegetable oil (sunflower seed oil, olive oil and apricot seed oil) olive leaf, hemp seed, and constituents of Shiunko (deoxyshikonin, β-βdimethylacrycrylshikonin, isobutylshikonin, shikonin) by Pseudozyma.

Korean Patent No. 10-2459871 is directed to a fermented oil—specifically, fermentation of turmeric and a vegetable oil (sunflower seed oil, olive oil, safflower oil, green tea seed oil, argan kernel oil) by Pseudozyma—and its use in cosmetics.

Korean Patent No. 10-2451466 teaches the fermentation of Paeonia lactiflora (Chinese Peony) and a vegetable oil (from the group sunflower seed oil, olive oil, safflower seed oil, green tea seed oil, camelia seed oil, argan kernel oil) by a Pseudozyma microorganism.

Environmental exposures (e.g., to pollutants, both indoor and outdoor, ultraviolet light and blue light, heat and cold,) diet, lack of sleep can result in swollen, lax skin, as well as fat accumulation in the under-eye area. Reduced production and quality (bundling) of collagen and other extra-cellular matrix proteins and resulting loss of elasticity are manifested as appearance of under-eye bags and dark circles. Compositions of the present invention respond to the need for targeted treatment to reduce the visible signs of chronological and extrinsic aging of the under-eye area.

### BRIEF DESCRIPTION OF THE FIGURES

FIG. 1 illustrates the protective effect of (a) fermented arnica oil (FAO) and (b) a complex containing FAO (in combination with (i) fermented Camellia sinensis seed oil, (ii) fermented oil of Angelica gigas root extract and Lithospermum erythrorhizon root extract root, and (iii) fermented oil of Glycyrrhia uralensis root extract versus (iii) non-fermented arnica oil (AO). AO did not protect cells from UVA-induced cytotoxicity. Round morphology of stressed/dying cells treated with AO (FIG. 1C) is noticeable in comparison with the normal elongated fiber-like morphology of cells incubated with FAO (FIG. 1A) and FAO Complex (FIG. 1B).

FIG. 2 are photographs of normal neonatal human dermal fibroblasts (nHDF) treated with FAO and stained with sulforhodamine B (eight wells to left) and nHDF treated with AO and stained with sulforhodamine B (eight wells to right). The intensity of the color is proportional to cell number/cell viability.

FIG. 3 illustrates the under-eye bag area measured in one clinical investigation of a topical composition containing an FAO Complex.

FIGS. 4-16 are images showing improvements in the appearance of the under-eye area.

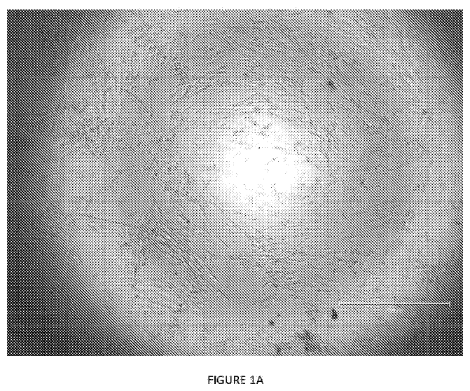

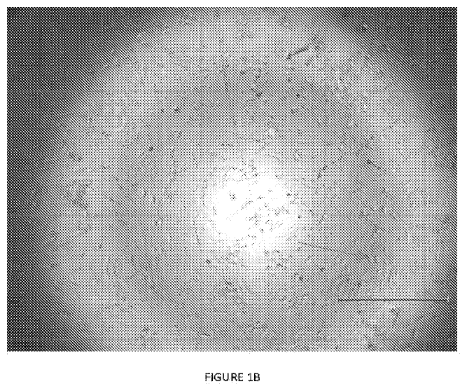

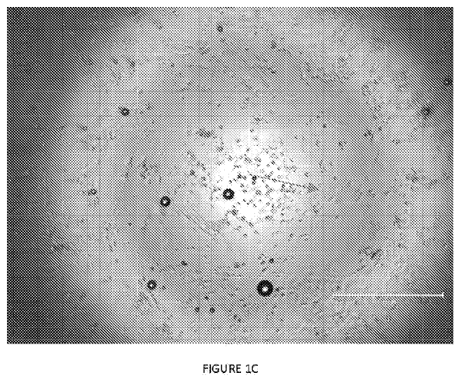

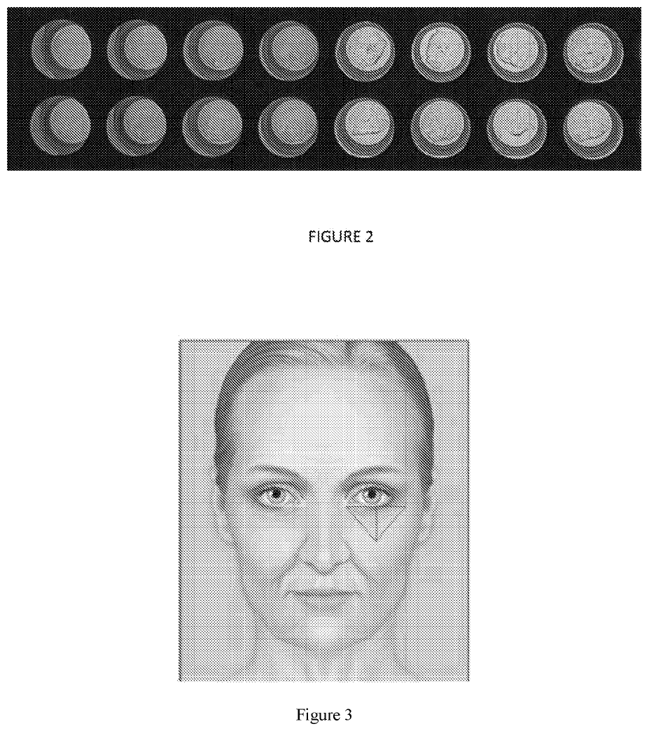

### SUMMARY OF THE INVENTION

An anti-inflammatory fermented arnica oil produced by fermenting oil extracted from the flowers of A. montana with a strain of Pseudozyma spp., preferably Pseudozyma epicula; compositions containing said fermented arnica oil or a complex containing said fermented arnica in combination with one or more secondary fermented oils that provides skin benefits including reduction of under-eye bag area, reduction of dark circles and/or puffiness in the under-eye area, increased skin brightness, increased skin hydration, reduced transepidermal water loss, improved skin barrier integrity.

### DETAILED DESCRIPTION OF THE INVENTION

Fermented arnica oil (FAO) is produced by fermenting oil extracted from the flowers of A. montana with a strain of Pseudozyma. A preferred Pseudozyma strain is Pseudozyma epicola.

In the present invention, an FAO complex is a combination of FAO (described above) and at least one, preferably at least two, more preferably at least three secondary fermented oils.

The secondary fermented oil(s) is/are produced by fermenting a botanical extract (e.g., an oil or root extract) with a Pseudozyma strain, preferably Pseudozyma epicola.

The botanical extract may be, and preferably is, selected from the group consisting of: Angelica Gigas/Lithospermum Erythrorhizon Root Extract; Artemisia Princeps Extract; Licorice Root Extract, preferably Glycyrrhia Glabra or Glycyrrhia Uralensis; Leonurus Sibiricus Extract; Panax Ginseng Root Extract; Saururus Chinensis Extract; Camellia Japonica Seed Oil; Carthamus Tinctorius Seed Oil; Argania Spinosa Kernel Oil; Camellia Sinensis Seed Oil.

Among the secondary fermented oils, the following are preferred: Angelica Gigas Root Extract and Lithospermum Erythrorhizon Root Extract; Camellia Sinensis Seed Oil; and Glycyrrhia Uralensis Root Extract.

Compositions of the present invention may, and preferably do contain, at least one secondary fermented botanical extract filtrate, produced by fermentation by Pseudozyma spp., preferably Pseudozyma epicola.

In certain embodiments, the at least one secondary fermented botanical extract is Fermented Green Tea Seed Oil—namely, a filtrate of the extract of the product obtained by the fermentation by Pseudozyma epicola of Camellia Sinensis Seed Oil, Glucose, Glycine Soja (Soybean) Flour, Malt Extract, and Yeast Extract.

In other embodiments, the at least one secondary fermented botanical extract is a Fermented Oil of Licorice Root Extract—namely, a filtrate of the extract of the product obtained by the fermentation by Pseudozyma epicola of Prunus Armeniaca (Apricot) Kernel Oil, Olea Europaea (Olive) Fruit Oil, Prunus Amygdalus Dulcis (Sweet Almond) Oil, Helianthus Annuus (Sunflower) Seed Oil, and Glycyrrhiza Glabra (Licorice) Root Extract.

In still other embodiments, the at least one secondary fermented botanical extract is a Fermented Oil of Angelica Gigas Root Extract and Lithospermum Erythrorhizon Root Extract—namely, a filtrate of the extract of the product obtained by the fermentation by Pseudozyma epicola of Prunus Armeniaca (Apricot) Kernel Oil, Olea Europaea (Olive) Fruit Oil, Helianthus Annuus (Sunflower) Seed Oil, Prunus Amygdalus Dulcis (Sweet Almond) Oil, Angelica Gigas Root Extract, and Lithospermum Erythrorhizon Root Extract.

In further embodiments, the at least one secondary fermented botanical extract is Fermented Sunflower Seed Oil—namely, a filtrate of the extract of the product obtained by the fermentation of Helianthus Annuus (Sunflower) Seed Oil by Pseudozyma epicola.

In one preferred embodiment, the at least one secondary fermented botanical extract is (i) Fermented Green Tea Seed Oil fermented by Pseudozyma epicola as described above and (ii) one fermented oil fermented by Pseudozyma epicola as described above selected from the group consisting of (a) Fermented Oil of Licorice Root Extract, (b) Fermented Oil of Angelica Gigas Root Extract and Lithospermum Erythrorhizon Root Extract or (c) Fermented Sunflower Seed Oil.

In another preferred embodiment, the at least one secondary fermented botanical extract is (i) Fermented Oil of Licorice Root Extract fermented by Pseudozyma epicola as described above and (ii) one fermented oil fermented by Pseudozyma epicola as described above selected from the group consisting of (a) Fermented Oil of Angelica Gigas Root Extract and Lithospermum Erythrorhizon Root Extract or (b) Fermented Sunflower Seed Oil.

In yet another preferred embodiment, the at least one secondary fermented botanical extract is (i) Fermented Oil of Angelica Gigas Root Extract and Lithospermum Erythrorhizon Root Extract and (ii) Fermented Sunflower Seed Oil fermented by Pseudozyma epicola, each as described above.

In one even more preferred embodiment, the at least one secondary fermented botanical extract is (i) Fermented Green Tea Seed Oil fermented by Pseudozyma epicola as described above and (ii) two fermented oils fermented by Pseudozyma epicola as described above selected from the group consisting of (a) Fermented Oil of Licorice Root Extract, (b) Fermented Oil of Angelica Gigas Root Extract and Lithospermum Erythrorhizon Root Extract or (c) Fermented Sunflower Seed Oil.

In another even more preferred embodiment, the at least one secondary fermented botanical extract is (i) Fermented Oil of Licorice Root Extract and (ii) Fermented Oil of Angelica Gigas Root Extract and Lithospermum Erythrorhizon Root Extract and (iii) Fermented Sunflower Seed Oil fermented by Pseudozyma epicola, each as described above.

In an especially preferred embodiment, the at least one secondary fermented botanical extract is (i) Fermented Green Tea Seed Oil fermented by Pseudozyma epicola, (ii) Fermented Oil of Licorice Root Extract fermented by Pseudozyma epicola, (iii) Fermented Oil of Angelica Gigas Root Extract and Lithospermum Erythrorhizon Root and (iv) Fermented Sunflower Seed Oil, each as described above.

In embodiments of the present invention directed to an FAO complex, the ratio of FAO to at least one secondary fermented oil can be, and preferably is, 2:1.

In certain embodiments in which the FAO complex is comprised of two secondary fermented oils, the ratio of the secondary fermented oils can be, and preferably is, 1:1.

In other embodiments in which the FAO complex is comprised of two secondary fermented oils, the ratio of FAO to the secondary fermented oils can be, and preferably is, 1:1.

In still other embodiments in which the FAO complex is comprised of three secondary fermented oils, the secondary fermented oils can be, and preferably are, present in a ratio of 1:1:1.

FAO complexes can, and preferably do, contain fermented oils—FAO and one or more secondary fermented oil(s)—and at least one non-fermented carrier oil. Preferably the non-fermented carrier oil is selected from the group consisting of Prunus Armeniaca (Apricot) Kernel Oil; Olea Europaea (Olive) Fruit Oil; Helianthus Annuus (Sunflower) Seed Oil; Carthamus Tinctorius (Safflower) Seed Oil; Prunus Amygdalus Dulcis (Sweet Almond) Oil.

The ratio of fermented oils—FAO and secondary fermented oil(s)—to non-fermented carrier oil(s) can range from 1:1 to 1:10 and is preferably about 1:4.

After the fermentation step, the process for producing the above-described fermented oils preferably includes a centrifugation and/or purification step.

Disclosed below are methods for reducing expression of genes that control or produce inflammation in human skin cells and/or skin tissue (including human skin substitutes from MatTek Corp.) by application of fermented arnica oil or a complex containing fermented arnica oil and methods for increasing the expression of genes associated with skin barrier integrity.

A first aspect of the present invention is directed to a method for protecting skin cells—specifically, normal neonatal human dermal fibroblasts—from UV-induced damage by application of FAO, as quantified in terms of cell morphology, cell viability and antioxidant protection (Lipo-ORAC assay).

Normal neonatal human dermal fibroblasts (nHDF) were plated in a 96-well format and cultured in Dulbecco's Modified Eagle's Medium (DMEM)/10% bovine calf serum (BCS) medium until the exponential growth phase was reached. Culture medium was replaced by phosphate buffered saline (PBS). Three test materials—non-fermented arnica oil (AO), fermented arnica oil (FAO), and a complex containing FAO and three additional fermented oils—green tea, Shiunko and licorice (FAO Complex)—were added on top of nHDF cells at 10% of total volume. Cells were UVA-irradiated at 4.5 mW/cm² for 5 minutes with UVA radiation (UVA-28T; Ultra-Lum; Sunnyvale, CA). The plate was returned to the incubator for 24 hours. Cells were fixed in cold trichloroacetic acid solution (10% in PBS) and stained with sulforhodamine B, a stain binding to cytoskeletal proteins, such as actin filaments, proportionally to cell numbers. Voigt W. “Sulforhodamine B Assay and Chemosensitivity.” Methods in Molecular Medicine 2005; 110: 39-48.

After rinsing three times with 1% acetic acid, the plate was dried, and cells were photographed with the EVOS imaging system (Thermo Fisher Scientific, Waltham, MA).

Sulforhodamine B was extracted in a PBS solution and quantified at 575 nm in Spectramax 190 Microplate Reader (Molecular Devices; Sunnyvale, CA). Statistical significance was assessed using the double tailed t-test. A threshold statistical significance was set at p=0.05 and ≥20% difference compared to control (distilled water).

Superior antioxidant protection (Lipo-ORAC) of FAO versus AO was observed in terms of Trolox Equivalents (TE) (moles TE/ml) based on the method of Huang (2002). Dilutions of test materials were prepared in an aqueous solution comprised of 50% acetone, 7% (2-Hydroxypropyl)-Beta-Cyclodextrin. Distilled water was the negative control; —Trolox served as a positive control and was used to prepare a standard curve for quantification of the measured ORAC values. Fluorescence was measured using Applied Biosystems CytoFluor Series 4000 Multi-Well Fluorescence Reader (Thermo Fisher, Waltham MA).

ORAC values were calculated from a regression equation of net areas under a fluorescence decay curve using the following formula: Area Under the Curve (AUC)=1+(Point 1+Point 2+Point 3+ . . . +Point 15)/Point 1.

Fermented arnica oil (FAO) provided 8.6 times more (860% increase) antioxidant protection versus non-fermented arnica oil (AO).

| Test Material | μmoles TE/ml |
|---|---|
| FAO | 2915 |
| AO | 338 |
| Distilled H₂O (Negative control) | 0 |
| Trolox (Positive control) | 4471 |

A second aspect of the present invention is directed to methods for reducing the expression of NF-κB-controlled genes by application of FAO and FAO Complex.

Three test materials—AO, FAO, FAO Complex—were evaluated for their ability to modulate NF-κB activity in CHO cells in a NF-κB Reporter assay (described below).

Test materials were not water-soluble and were dissolved and diluted in caprylic/capric triglyceride (CCT). Cell viability was confirmed after 18 hours of incubation with test materials, using the RealTime-Glo™ MT Cell Viability assay (Promega, Corp.; Madison, WI), an assay that determines the number of viable cells in culture by measuring the reducing potential of cells, and thus metabolism.

NF-κB Reporter is a mixture of an inducible NF-κB-responsive Luciferase construct and a constitutively expressing Renilla construct in a ratio of 40:1. The NF-κB-responsive Luciferase construct encodes the firefly luciferase reporter gene under the control of a minimal cytomegalovirus (CMV) promoter with tandem repeats of the NF-κB transcriptional response element. This construct measures both increases and decreases in transcriptional regulatory activity of NF-κB. The constitutively expressing Renilla construct encodes the Renilla luciferase reporter gene under the control of a CMV enhancer/promoter, which acts as an internal control for normalizing transfection efficiencies and monitoring cell viability. The number of response elements as well as the intervening sequence between response elements was optimized to maximize signal/noise ratio.

CHO cells were seeded at 50,000 cells/well in OPTI-MEM (Thermo Fisher Scientific, Waltham, MA)+1% MEM non-essential amino acid solution (Sigma Aldrich, St. Louis, MO) in a 96-well black wall plate and grown overnight. The following day, the CHO cells were transfected by (i) NF-κB Reporter Assay firefly/renilla luciferase constructs (Catalog #60616; BPS Bioscience, Inc.; San Diego, CA) combined with (ii) Plasmid Transfection Reagent (Santa Cruz Biotechnology, Inc.; Dallas, TX) for 6 hours. After transfection, cell culture medium was changed to DMEM/1% CS. Test materials were then added and their effect on NF-κB induction was quantified 18 hours later in the presence of 20 ng/ml TNF-α (added at the same time as test articles), using the Dual-Luciferase Reporter Assay System (Catalog #E1960; Promega Corp.; Madison, WI).

Signal quantification was obtained with Luminoskan Ascent Microplate Luminometer (Thermo Fisher Scientific; Waltham, MA) certified as “Dual-Luciferase Assay-Ready” based on DLReady™ validation by Promega Corp. (Madison, WA).

5% CCT in cell culture media was observed to begin to inhibit cellular metabolism. Accordingly, the Reporter Assay was performed at 0.5% CCT. At three concentrations tested—10 mg/ml; 2 mg/ml; and 0.4 mg/ml the test materials were not cytotoxic.

P values representing statistical significance were calculated using the Student t-test, and threshold of statistical significance for microplate fluorometry readings was set at p=0.100 and ≥20% difference compared to a water control.

At 10 mg/ml, FAO reduced expression of NF-κB-controlled genes by 62% more than AO (p=0.077).

A third aspect of the present invention is directed to a method for decreasing the expression TNF-α by topical application of FAO as measured by real-time polymerase chain reaction (RT-PCR).

EpiDerm® skin substitute tissues (Catalog #EPI-212; Lot #37834) were obtained from MatTek Corp. (Ashland, MA). After 6-hour equilibration in a cell culture incubator, FAO was applied to the surface of the EpiDerm® skin substitute tissues with a positive displacement pipette at 5 μl/tissue and incubated for 24 hours. Water served as a negative control. After an initial 24-hour incubation, a pro-inflammatory effector—lipopolysaccharide (LPS) (Catalog #0560535-2; Cayman Chemical, Co.; Ann Arbor, MI)—was added to the tissue culture medium at 10 μg/ml, which was incubated for 24 hours. R. Gvirtz et al. “Kinetic Cytokine Secretion Profile of LPS-Induced Inflammation in the Human Skin Organ Culture.” Pharmaceutics. 2020 Mar. 25; 12(4):299.

At the end of the LPS incubation, skin substitute tissues were rinsed. RNA was extracted and purified with NucleoSpin kit from Macherey Nagel (Bethlehem, PA; Catalog #740955) using a QiaCube Connect robotic station (Qiagen, Germantown, MD). Purified total RNA was assessed at 260 nm and 280 nm with NanoDrop Lite (Thermo Fisher Scientific, Waltham, MA).

cDNA was prepared using AzuraQuant cDNA kit (Azura Genomics, Raynham, MA). Expression of TNF-α was measured by real-time quantitative PCR with BioRad iCycler iQ Detection System using PCR primers from Realtimeprimers (Elkins Park, PA) and AzuraView GreenFast qPCR Blue Mix LR (Azura Genomics). After normalization to housekeeping genes (18S and GAPDH), the ΔΔCt method was used for quantification of level of gene expression. Ct (cycle threshold) is defined as the number of replication cycles required for the fluorescent signal to exceed the background level. Ct levels are inversely proportional to the amount of target nucleic acid in the sample (i.e., the lower the Ct level, the greater the amount of target nucleic acid in the sample).

Treatment of cells with FAO decreased TNF-α gene expression by 60%. This result was strongly “directional”; but of moderate statistical significance (p=0.15).

A fourth aspect of the present invention is directed to a method for increasing the expression of a gene that codes for a protein that provides skin barrier protection, preferably loricrin, by topical application of FAO as measured by RT-PCR.

After 6-hour equilibration in a cell culture incubator, FAO was applied to the surface of the EpiDerm® skin substitute tissues with a positive displacement pipette at 5 μl/tissue and incubated for 24 hours. Water served as a negative control. After an initial 24-hour incubation, lipopolysaccharide (LPS), a pro-inflammatory effector, was added to the tissue culture at 10 μg/ml, which was incubated for 24 hours. At the end of the LPS incubation, skin substitute tissues were rinsed. RNA was extracted and purified with NucleoSpin kit from Macherey Nagel using a QiaCube Connect robotic station. Purified total RNA was assessed at 260 nm and 280 nm with Thermo Fisher NanoDrop Lite (Thermo Fisher Scientific, Waltham, MA). cDNA was prepared using AzuraQuant cDNA kit. Expression of loricrin was measured by real-time quantitative PCR with BioRad iCycler iQ Detection System using PCR primers from Realtimeprimers and AzuraView GreenFast qPCR Blue Mix LR. The ΔΔCt method was used for quantification of level of gene expression after normalization to housekeeping genes (18S and GAPDH).

In skin tissue substitutes treated with FAO, expression of the skin barrier protective gene loricrin was statistically significant; an increase of 290% was observed with p-value of 0.05 based on the Student t-test.

A fifth aspect of the present invention is directed to a method for decreasing the expression of at least one, preferably at least two, and still more preferably three inflammatory interleukin cytokines selected from the group consisting of IL-1α, IL-1β, and IL-8 by topical application of FAO Complex as measured by sandwich enzyme-linked immunosorbent assay (ELISA), an analytical technique that measures antigen between two layers of antibodies (capture antibody and detection antibody).

EpiDerm® skin substitute tissues were treated with FAO Complex at 5 μl/tissue and incubated for 24 hours. LPS was then added to the tissue culture at 10 μg/ml, which was incubated for 24 hours. At the end of the LPS incubation, levels of interleukins IL-1α, IL-1β, and IL-8 in the tissue conditioned medium were quantified by sandwich ELISA using the following antibody pairs: IL-1α DuoSet® Catalog #DY200 (R&D Systems, Inc.; Minneapolis, MN):IL-1b: DuoSet® Catalog #DY201 (R&D Systems); IL-8: Catalog #s BioLegend® Catalog #s 511403 and 511502 (BioLegend; San Diego, CA)

At levels tested, FAO Complex exhibited statistically significant downregulation of each of the three interleukin genes tested. Statistical significance in this investigation was defined as greater than 50% variation from a water control (p value<0.05) calculated using double-tailed t-test.

| Gene | Percent Control | p value |
|---|---|---|
| IL-1 α | 75.4 | 0.046 |
| IL-1β | 82.2 | 0.04 |
| IL-8 | 63.5 | 0.01 |

A sixth aspect of the present invention is directed to topical compositions suitable for application to human skin, including to enhance or alter appearance, including accentuating certain features (eyelashes, eyebrows, lips) adding color to a person's face, concealing blemishes or imparting benefits to the skin (e.g., improving hydration, reducing trans-epidermal water loss, reducing laxity) that contain FAO or FAO Complex in accordance with the first, second, third, fourth or fifth aspects of the invention. Topical compositions can be in the form of a single-phase system (e.g., anhydrous) or a multi-phase system (e.g., emulsion) and can be a serum, gel, lotion, cream, or pressed powder.

In these embodiments, FAO can be included in topical compositions at a concentration (weight/weight) of from about 0.01% to about 100%, preferably from about 0.1% to about 10%.

In these embodiments, an FAO Complex can be included in topical compositions at a concentration (weight/weight) of from about 0.01% to about 20%, preferably from about 0.1% to about 10%.

Topical compositions in accordance with the sixth aspect of the present invention can, and preferably do contain, one or more mucopolysaccharides, preferably Hyaluronic Acid (“HA”)—a mucopolysaccharide formed by bonding N-acetyl-D-glucosamine with glucuronic acid—or its salts, esters, hydrolysates, polymers or other compounds formed by reaction with Hyaluronic Acid moiety (collectively “HA moieties”).

Preferred, but non-limiting, HA moieties include: (a) Sodium Hyaluronate—the sodium salt of HA; (b) Potassium Hyaluronate—the potassium salt of HA; (c) Hydrolyzed Hyaluronic Acid—the hydrolysate of HA derived by acid, enzyme or other method of hydrolysis; (d) Hydrolyzed Sodium Hyaluronate—the hydrolysate of Sodium Hyaluronate derived by acid, enzyme or other method of hydrolysis; (e) Sodium Acetylated Hyaluronate—the acetyl ester of Sodium Hyaluronate; (f) Sodium Hyaluronate Crosspolymer—the sodium salt of a polymer of HA crosslinked with vinyl sulfone; and (g) Hydroxypropyltrimonium Hyaluronate—a quaternary ammonium compound that conforming to the formula:

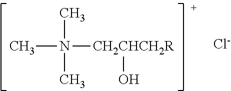

where R represents a HA moiety.

In certain embodiments of the sixth aspect of the present invention, at least two HA moieties are present in the topical composition; in other embodiments, at least three HA moieties are presently; in still other embodiments, at least four HA moieties are present. In further embodiments of the sixth aspect of the present invention, at least five HA moieties are present in the topical composition. Six and seven HA moieties may be present in the topical composition.

In addition to one or several HA moieties, topical compositions in accordance with the sixth aspect of the present invention can contain one or more skin benefit ingredients selected from the group consisting of botanical or algal extracts, antioxidants, anti-inflammatory agents, vitamins (e.g., tocopherol, niacinamide); silicones (fluids, elastomers, co-polymers, cross-polymers); film-forming polymers and/or rheology modifiers; humectants; emollients; skin conditioning agents; emulsifiers; iron oxides and/or titanium oxide. Solanum Lycopersicum (Tomato) Fruit Extract Non-limiting but preferred botanical or algal extracts that may be included in compositions include Solanum Lycopersicum (Tomato) Fruit Extract, Dunaliella Salina Extract, Coleus Forskohlii Root Extract, Pseudanabaena Galeata Extract, and Aloe Barbadensis Leaf Extract.

In certain embodiments, the FAO complex contains (in addition to fermented arnica oil and at least one secondary fermented oil, one or both of said oils being the product of fermentation by Pseudozyma epicola) one or both of Solanum Lycopersicum (Tomato) Fruit Extract and/or Dunaliella Salina Extract.

Topical compositions in accordance with the sixth aspect of the present invention can provide multiple skin benefits, including reduction in the appearance of under-eye bags, reduction in the appearance of under-eye puffiness, increased under-eye brightness, improved skin barrier protection as evidenced by one or both of improved skin hydration and/or decreased transepidermal water loss (TEWL), reduced appearance of redness (erythema), reduced appearance of mottling, improved (more even) skin tone.

The following examples of a composition (a concealer) containing an FAO Complex of the present invention are illustrative. The scope of the appended claims is not to be limited to the examples.

The effects of a concealer containing an FAO Complex and at least two HA moieties was evaluated on under-eye bag area, under-eye puffiness, and dark circles was evaluated in a 4-week (28 day) clinical study involving 15 healthy Caucasian subjects aged between 20 and 60 years with visible under-eye bags. Criteria for inclusion (eligibility to participate) in study include the following: (i) willingness to (a) maintain the assigned skin care regimen for study duration (b) follow directions regarding product usage and (c) attend all required study visits; (ii) no plans to modify/change lifestyle during regimen (e.g., weight loss; eating, drinking, or smoking habits; level/amount of sport activities). Exclusion criteria are not eligible to participate in the study: (i) pregnant or planning to conceive; (iii) diagnosed with a skin condition (e.g., psoriasis, eczema); (iv) known allergenicity/hypersensitivity to a skin care product.

At four time points, clinical photographs under standardized conditions (air-conditioned room at 20 °C (±2 °C) and controlled humidity (50% RH±5%) after an acclimatization period of about 30 minutes) were taken to evaluate under-eye bag area: (i) at baseline on Study Day 0 (T₀); (ii) after one week, measured on Study Day 7 (T_{7d}); (iii) after three weeks, measured on Study Day 14 (T_{14d}); and (iv) after four weeks, measured on Study Day 28 (T_{28d}). Using VISIA® Skin Analysis System (Canfield Scientific, Inc., Parsippany, NJ, USA), two measurements were taken: eye width (distance between the inner and external corner) was measured (in mm) on frontal view; eye bag height was measured (in mm) on side view. Eye bag width multiplied by eye bag height, divided per 2, directly correlates with under-eye bag area (in mm²). See FIG. 3.

The following statistically significant improvements (all p<0.001) were observed in under-eye bag area:
after one week of once daily application −5.0% change (% Δ (T7_{d}−T₀);
after two weeks of once daily application −8.5% change (% Δ (T_{14d}−T₀);
after four weeks of once daily application −₁₂0.0% change (% Δ (T_{28d}−T₀).

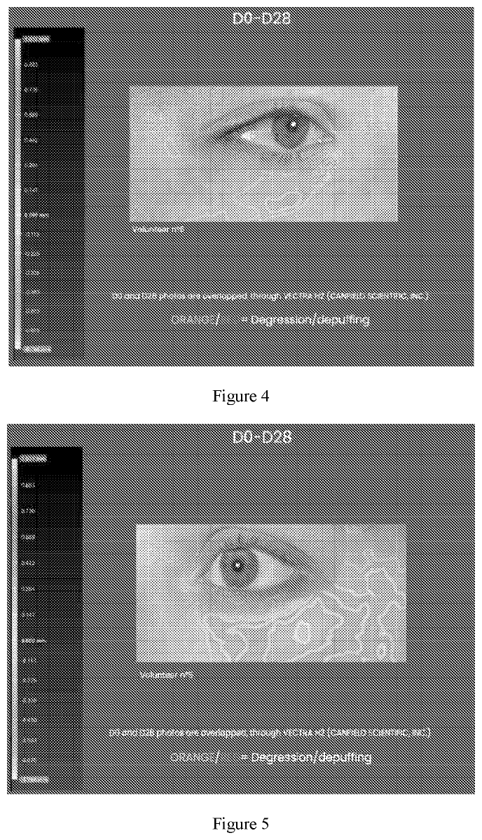

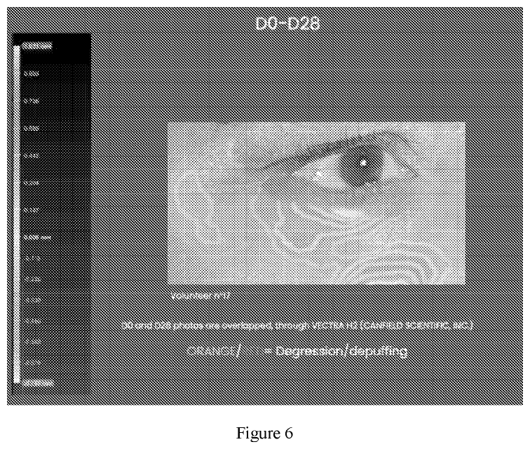

At two time points, —at baseline on Study Day 0 (T₀); and at study end, after 4 weeks (T28d)—three consecutive photographs were taken of each subject from three angles using VECTRA H2 System (Canfield Scientific, Inc., Parsippany, NJ, USA): the first photograph was taken 30 cm below 45 degrees on the right side of the study participant's face; the second photograph was taken with the camera in front of the participant's face; the third picture was taken on the left side of the participant's face at 30 cm below 45 degrees. A computer connected to the camera merged the three photographs into a single 3D photo using VAM software (Canfield Scientific, Inc.) and outputs contour mapping images showing improvements (reduced puffiness). See FIGS. 4-6.

At three time points—baseline, 15 minutes after initial application, and after two weeks of once-daily application of a composition of the present invention, changes in the appearance of puffiness under the eyes (“brightening”) was evaluated by an expert grader using a ten-point Visual Analogue Scale (VAS): 0=no appearance of under-eye puffiness; 1-3=mild appearance of under-eye puffiness; 4-6=moderate appearance of under-eye puffiness; 7-9=severe appearance of under-eye puffiness.

At the same three time points, changes in the appearance of brightness under the eyes (“brightening”) were evaluated by an expert grader using a ten-point Visual Analogue Scale (VAS): 0=complete under-eye brightness; 1-3=severe appearance of under-eye brightness; 4-6=moderate appearance of under-eye brightness; 7-9=mild to no appearance of under-eye brightness.

Statistically significant improvements (p<0.001) expressed as negative mean percent change from baseline were observed after 15 minutes and after 2 weeks in the appearance of both under-eye puffiness and under-eye brightness from baseline.

| | Under-eye Puffiness | | Under-eye Brightness | |
|---|---|---|---|---|
| Parameter | 15 minutes | 2 weeks | 15 minutes | 2 weeks |
| Mean % Change from | −12.55% | −12.55% | −16.3% | −4.63% |
| Baseline | | | | |

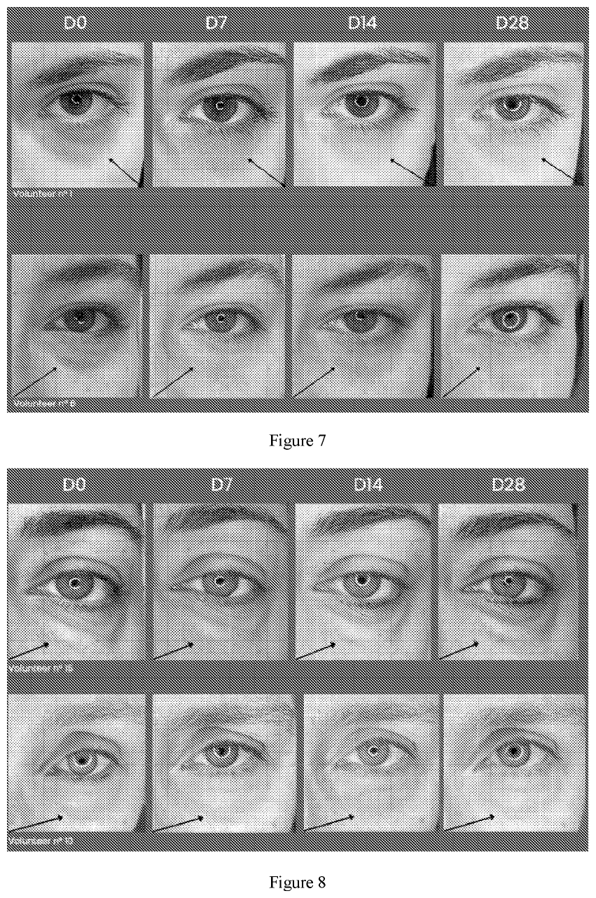

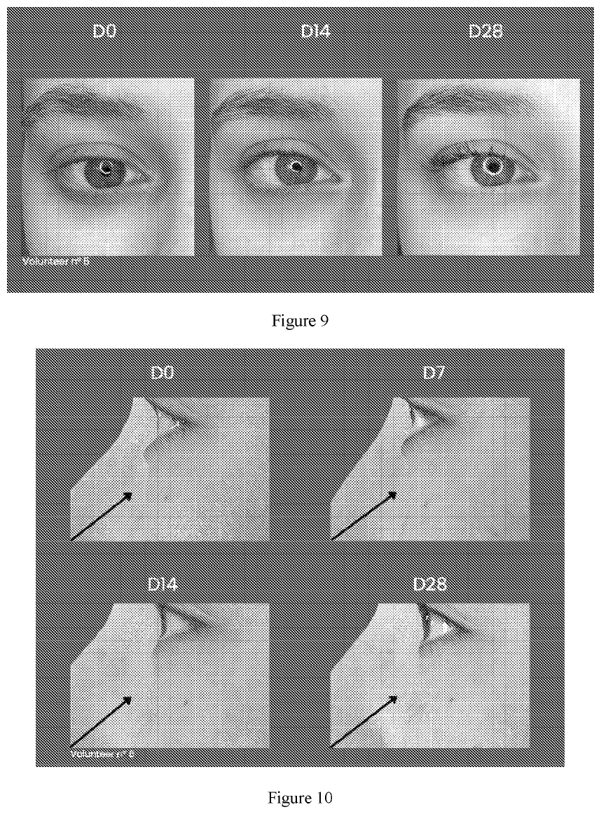

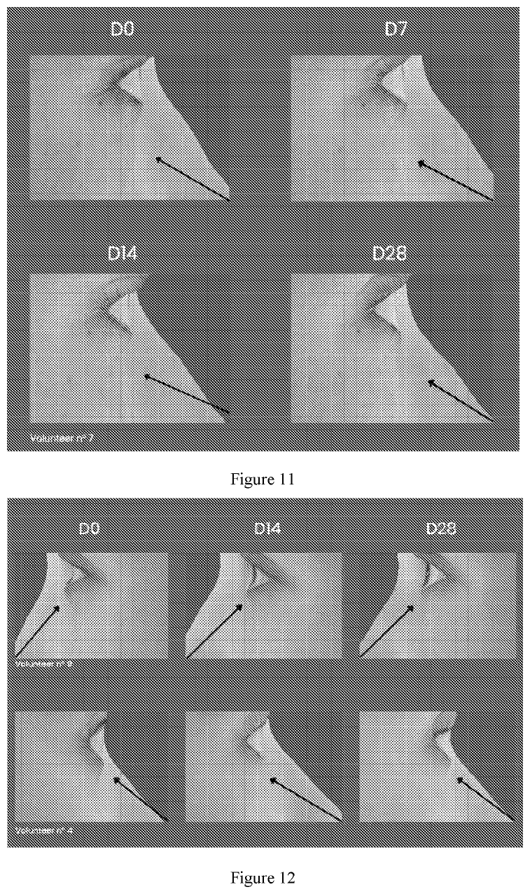

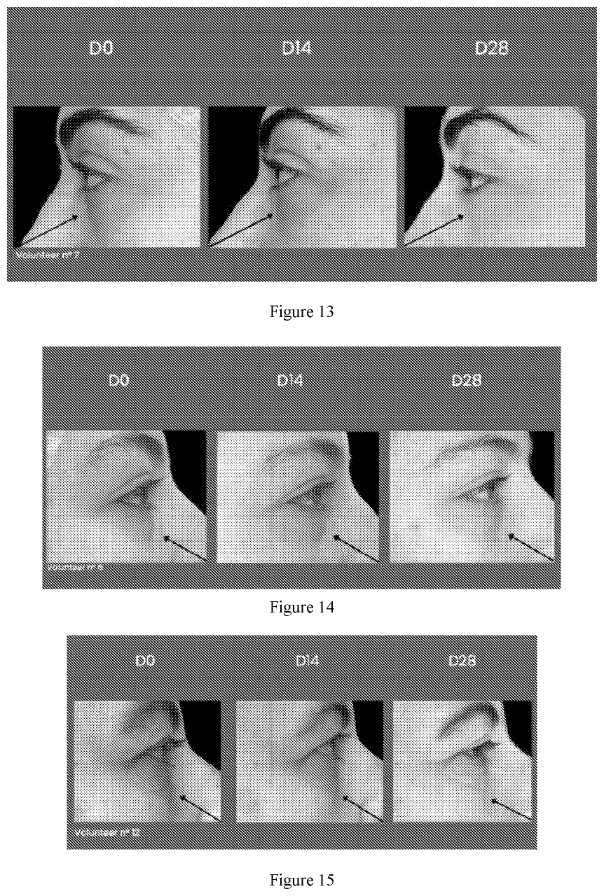

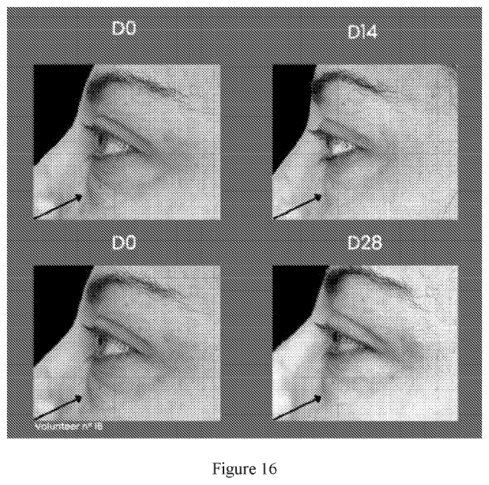

Improvements in the appearance of the under-eye area, as evidenced by reduction in one or more of under-eye bag area, under-eye puffiness, under-eye dark circles and/or increase in under-eye brightness are shown in FIGS. 7-16.

Skin hydration reflects water content of the skin's outermost layer, the stratum corneum (SC), and is evaluated with a Corneometer, a device that measures hydration levels based on dielectric properties of the SC at a depth of approximately 10-20 μm. After a single application of the concealer composition of the present invention, an increase of more than 25% in skin hydration (as assessed by Corneometer) was reported in 36 of 37 study participants.

The efficacy of topical compositions containing FAO or an FAO Complex can further confirmed using one or more of the following test methods known to the person have ordinary skill in the art of developing cosmetic and dermatologic products.

Improvements in brightness and reduction in visible appearance of mottling and redness can be demonstrated in terms of digital image analysis of changes in L* and a* vs. baseline in accordance with the CIE 1976 (L*a*b*) system. A. R. Robertson, “The CIE 1976 color-difference formulae,” Color Res. Appl. Vol. 2, pp. 7-11 (1977). L* is measured from 0 to 100, where “100” represents white (a “perfect reflecting diffuser”); and “0” represents black. a* and b* are chromaticity coordinates on two axes: red-green and blue-yellow. Positive a* indicates red; negative a* indicates green. Positive b* indicates yellow; negative b* indicates blue. See, support.hunterlab.com/hc/en-us/articles/203996325-CIE-L-a-b-Color-Scale-an07-96a (accessed on Sep. 23, 2022).

Reduction in visible redness (erythema) can be quantified on a VAS scale ranging from 0 to 10, where “0” represents no to very little erythema, possible with slight scaling, and “10” represents pronounced erythema, infiltration, possibly vesicles.

Reduction in skin dryness can be quantified on a VAS scale from 0 to 10, where “0” represents no or very little evidence of dryness, and “10” which represents severe flaking, peeling and/or fissures.

Reduction in visible mottling can be quantified on a VAS scale from 0 to 10, where “0” represents even skin tone, with no to very little mottling, and “10” represents a pronounced pattern of irregular marks, spots, streaks, blotches or patches of different shades or colors.

In addition to skin hydration (discussed above), rate of transepidermal water loss (TEWL) is a widely-used indicia for evaluating skin barrier function. Du Plessis, J et al. “International Guidelines for the In Vivo Assessment of Skin Properties in Non-Clinical Settings: Part 2.” Skin Res Technol. 2013 August; 19(3): 265-278.

Increased (e.g., improved) skin barrier function can be assessed in terms of trans-epidermal water loss (TEWL) using a Tewameter® (Courage+Khazaka electronic GmbH, Koln, Germany).

More particularly, a Tewameter® measures the rate of evaporation from the skin surface according to the formula:

$$\frac{dm}{dt} = - D \cdot A \cdot \frac{dp}{dx}$$

where
m=water transported (grams)
t=time (hours)
D=diffusion constant=0.0877 (mm Hg) (also expressed as g/h/m²)
A=surface area (m²)
p=atmospheric vapor pressure (mm Hg)
x=distance from skin surface to point of measurement (meters)

In assessing the skin barrier protective properties of the test product TEWL is measured on a test site (e.g., 2 cm²) marked on the lower lateral leg as follows. At least three Tewameter® measurements are taken: (i) prior to stripping (removal of the stratum corneum, resulting in increased TEWL); (ii) post-stripping; (iii) post application of the test product. Return of TEWL to a level approximately the same as baseline is indicative of improved skin barrier function.

1. An anti-inflammatory fermented arnica oil produced by fermenting oil extracted from the flowers of A. montana with a strain of Pseudozyma spp.

2. The anti-inflammatory fermented arnica oil of claim 1 wherein the strain of Pseudozyma is Pseudozyma epicola.

3. The anti-inflammatory fermented arnica oil of claim 2 that reduces expression of NF-κB.

4. An anti-inflammatory fermented arnica oil of any of claims 1-3 that reduces the expression of TNF-α.

5. A skin-barrier protective fermented arnica oil produced by fermenting oil extracted from the flowers of A. montana with a strain of Pseudozyma spp. that increases expression of loricrin.

6. An anti-inflammatory complex comprised of a fermented arnica oil according to any of claims 1-3 and further comprising at least one secondary fermented oil produced by fermenting a strain of Pseudozyma spp. with a botanical extract selected from the group consisting of (a) Angelica gigas root extract and/or Lithospermum erythrorhizon root extract; (b) Camellia sinensis seed oil; (c) Glycyrrhia uralensis root extract; and (d) Helianthusa seed oil.

7. The anti-inflammatory complex of claim 6 that reduces the expression of IL-1α, IL-1β, and IL-8.

8. A topical composition comprising (a) an anti-inflammatory fermented arnica oil of any of claims 1-3, (b) a skin-barrier protective fermented arnica oil of claim 4, (c) an anti-inflammatory complex of claim 6 or 7.

9. A topical composition of claim 8 further comprising hyaluronic acid or a hyaluronic acid moiety selected from the group consisting of (a) sodium hyaluronate; (b) potassium hyaluronate; (c) hydrolyzed hyaluronic acid; (d) hydrolyzed sodium hyaluronate; (e) sodium acetylated hyaluronate; (f) sodium hyaluronate crosspolymer; and (g) hydroxypropyltrimonium hyaluronate.

10. A method of improving the appearance of under-eye area by providing one or more skin benefits selected from the group consisting of (a) reducing under-eye bag area, (b) reducing under-eye puffiness, and/or (c) increasing under-eye brightness, comprising the step of applying to the under-eye area a topical composition of claim 8.
